(19) United States
(12) Patent Application Publication
(10) Pub. No.: US 2005/0163873 A1
(43) Pub. Date: Jul. 28, 2005

(54) Methods and formulations for treating glaucoma

(76) Inventor: Robert Ritch, New York, NY (US)

Correspondence Address: BROMBERG & SUNSTEIN LLP, 125 SUMMER STREET, BOSTON, MA, 02110-1618, US

(21) Appl. No.: 11/030,588

(22) Filed: Jan. 6, 2005

Related U.S. Application Data
(60) Provisional application No. 60/536,339, filed on Jan. 14, 2004.

(51) Int. Cl. A61K 35/78; A61K 31/714; A61K 31/525; A61K 31/385

(52) U.S. Cl. 424/752; 514/52; 514/440; 514/251; 424/728; 424/94.1; 514/565; 514/733; 424/746

(57) ABSTRACT

Embodiments of the invention are directed toward neuroprotective formulations that may prevent the progression of glaucoma by strengthening the resistance of nerve cells to damage. Some embodiments of the invention are directed toward neuroprotective formulations that may limit and prevent glaucomatous damage by blocking the mechanisms that lead to retinal ganglion cell death. Other embodiments of the invention involve specific component formulations and methods of using the formulations.

### RELATED APPLICATIONS

This application is a regular application and claims priority from U.S. provisional patent application No. 60/536,339 filed Jan. 14, 2004, the entire contents of which are hereby incorporated by reference herein.

### TECHNICAL FIELD

The present invention relates to methods and compositions for treating glaucoma, and more particularly to methods and compositions for reducing one or more non-intraocular pressure-dependent risk factors of glaucoma through neuroprotection.

### BACKGROUND ART

Glaucoma is a progressive optic neuropathy (a disease of the optic nerve) characterized by a specific pattern of optic nerve head and visual field damage. Damage to the visual system in glaucoma is due to the death of the retinal ganglion cells (RGCs), the axons of which comprise the optic nerve and carry the visual impulses from the eye to the brain. RGCs die in glaucoma by apoptosis, or programmed cell death. Glaucoma represents a final common pathway resulting from a number of different conditions that can affect the eye, many of which are associated with elevated intraocular pressure (IOP).

It is important to realize that elevated IOP is not synonymous with glaucoma, but rather is the most important risk factor we know of for the development and/or progression of glaucomatous damage. Indeed, glaucoma often progresses despite lowering of IOP to acceptable or normal levels. Therefore, a need exists for therapies that prevent or limit the damage due to glaucoma, independent of therapies that simply lower IOP.

### SUMMARY OF THE INVENTION

In some embodiments of the invention, a neuroprotective formulation for lowering non-intraocular pressure-dependent risk factors of glaucoma is presented. In one specific embodiment of the invention, a neuroprotective formulation includes ginkgo biloba extract, resveratrol, and pycnogenol. In a second specific embodiment of the invention, a neuroprotective formulation includes carnitine, methylcobalamin, and coenzyme Q10. In a third specific embodiment of the invention, a neuroprotective formulation includes ginseng extract and salvia militiorrhiza. A fourth specific embodiment of the invention is directed toward a neuroprotective formulation that includes ginko biloba extract and salvia militiorrhiza. A fifth specific embodiment of the invention is directed toward a neuroprotective formulation that includes carnitine and folic acid. In a sixth specific embodiment, a neuroprotective formulation includes pycnogenol, alpha-lipoic acid, and fish oil.

Other specific embodiments of the invention are directed toward neuroprotective. formulations that utilize the components from one of the six specific embodiments and include one or more components from at least one of the other specific embodiments.

Further embodiments of the invention utilize one of the specific embodiments previously discussed and include one or more of green tea catchins, quercetin, grape seed extract, alpha-tocopherol, choline, glutathione, N-acetyl-L-cysteine, taurine, zeaxanthine, lutein, citocoline, vitamin A, copper, magnesium, selenium, and zinc.

Additional embodiments utilize one of the specific embodiments previously discussed and include one or more of bilberry (Vaccinium myrtilis) extract, hawthorne (Crataegus spp.) extract, arctigenin (a lignanolide isolated from the bark of Torreya nucifera and Arctium lappa, for example), scrophularia extract (including, for example, iridoid glycosides and e-p-methoxycinnamic acid), forskolin (Coleus forskolii) extract, s-allylmercaptocysteine, and glucosamine.

Neuroprotective formulations consistent with embodiments of the invention may be in the form of one or more pills or liquid formulations. When more than one pill or liquid formulation is used in a neuroprotective formulation, the pills or liquid formulation may differ, in composition or quantity of a component, from each other.

Another embodiment of the invention is a method of preventing the progression of glaucoma in a patient. The method includes the steps of providing a neuroprotective formulation according to one of the specific embodiments previously discussed; and administering the neuroprotective formulation to the patient to reduce the risk of one or more non-intraocular pressure-dependent factors of glaucoma in a patient. The neuroprotective formulation may be in a dosage formulation. The method may include the step of administering the neuroprotective formulation on a scheduled basis; such administration may include administering one or more formulations according to the scheduled basis.

Another method consistent with an embodiment of the invention includes the steps of providing a neuroprotective formulation according to one of the specific embodiments previously discussed; and administering the neuroprotective formulation to the patient to strengthen the resistance of damage to nerve cells.

### DETAILED DESCRIPTION OF EMBODIMENTS

Unlike much of the prior art regarding compositions used to fight glaucoma, embodiments of the present invention are directed toward novel neuroprotective formulations, and methods of using such formulations, to reduce risk factors for glaucomatous damage besides those due to elevated IOP. These risk factors are referred to herein as non-IOP-dependent risk factors. The damage due to these risk factors is referred to as non-IOP-dependent damage. The most intensively investigated cause of non-IOP-dependent damage is the possibility of an insufficient blood supply to the optic nerve head and adjacent retina. Risk factors for this may include low blood pressure, orthostatic hypotension, nocturnal hypotension, atrial fibrillation, migraine, Raynaud's phenomenon, abnormally low intracranial pressure, autoimmune phenomena, and sleep apnea. Other risk factors associated with hemorheologic (flow properties of blood) abnormalities, such as increased erythrocyte agglutinability (tendency for red blood cells to stick to each other), decreased erythrocyte deformability (ability of the red blood cells to change shape so that they can squeeze into capillaries), increased serum viscosity, or increased platelet aggregability may also play a role.

Secondary degeneration refers to the spread of degeneration to apparently healthy neurons that escape the primary insult, but are adjacent to injured neurons and are thus exposed to the degenerative milieu that the latter create. Secondary degeneration is based on the finding that neuronal damage in the central nervous system may progress even when the primary cause of damage is alleviated. Neuronal death may be viewed as occurring in three steps: 1) axonal injury, 2) death of the injured neuron, and 3) injury and death of previously intact neurons through secondary degeneration. Neuroprotection refers to the postinjury protection of neurons that were initially undamaged or only marginally damaged by a particular insult, but are at risk from toxic stimuli released by damaged cells which cause secondary degeneration. In the latter instance, neuroprotection includes neural rescue, which refers to the restoration of viability to neurons which are already damaged. Neuroprotection is useful even when the exact cause of a disorder is undefined, as the therapy occurs at the level of the dying cells and not at the initial injury.

Neuroprotective strategies and pharmaceutical agents have been initiated in the treatment of numerous disorders of the central and peripheral nervous systems, including trauma, epilepsy, stroke, Huntington's disease, amyotrophic lateral sclerosis, and AIDS dementia. In the context of applying such strategies to treat glaucoma, an advantage of neuroprotection may be to limit and prevent glaucomatous damage by blocking the mechanisms which lead to RGC death, independent of lowering IOP. However, some embodiments of the invention may be directed toward preventing the progression of glaucoma by strengthening the resistance of nerve cells to damage, regardless of whether such damage is related or unrelated to IOP.

Many categories of both natural and synthetic compounds have been reported to have neuroprotective activity. These include not only antioxidants, NMDA receptor antagonists, inhibitors of glutamate release, calcium channel blockers, polyamine antagonists, and nitric oxide synthase inhibitors, but cannabinoids, aspirin, melatonin, and vitamin B-12. However, there is a lack of clinical trials and experience of using such compounds, or combinations of such compounds, as neuroprotective agents for glaucoma. Indeed, there is no currently proven clinically effective neuroprotective agent for the treatment of glaucoma. Given the success of certain compounds, and combinations of such compounds, in providing neuroprotective activity in other contexts, some embodiments of the present invention are directed toward novel formulations and methods of treating and preventing glaucoma using neuroprotective strategies.

The following compounds, and uses thereof singularly or in combination, represent embodiments of the present invention which may have benefits in the treatment of glaucoma and offer the possibility of neuroprotective activity.

Alpha-Lipoic Acid

Alpha-lipoic acid is a cofactor in mitochondrial dehydrogenase complexes. When administered exogenously, it has powerful antioxidant properties, which include free radical scavenging, metal chelation and regeneration of other antioxidants. Lipoic acid decreases iron uptake from transferrin and reduces the size of the highly reactive Fe pool in the cytoplasm of cells of the lens, changes associated with increased cell resistance to oxidative damage. Alpha-lipoic acid may help to prevent or slow progression of cataract.

Increases in leukostasis/monocyte adhesion to the capillary endothelium and decreased retinal blood flow are implicated in the pathogenesis of diabetic retinopathy. In diabetic rats, treatment with the antioxidant alpha-lipoic acid normalized the amount of leukostasis but not retinal blood flow, while treatment with D-alpha-tocopherol prevented the increases in leukostasis and decreases in retinal blood flow in diabetic rats.

Fish Oil and Omega-3 Fattyacids

Omega-3 fatty acids, such as docosahexaenoic acid (DHA) and eicosapentaeneoic acid (EPA) have major health benefits. DHA is thought to play an important role in providing an adequate environment for conformational rhodopsin changes and in modifying the activity of retinal enzymes in photoreceptor cells. Decreased retinal DHA content affects visual function in the monkey.

Oxidative damage induces apoptosis in retinal neurons during their early development in culture and suggests that the loss of mitochondrial membrane integrity is crucial in the apoptotic death of these cells. DHA activates intracellular mechanisms that prevent this loss and by modulating the levels of pro- and antiapoptotic proteins of the Bcl-2 family, selectively protects photoreceptors from oxidative stress.

DHA is effective intraperitoneally in protecting the retina against transient retinal ischemia induced by elevated intraocular pressure. Oral DHA can partially counteract retinal neurotoxicity induced by kainic acid. In ischemia-reperfusion injury, DHA protects against cell death probably by inhibiting the formation of hydroxyl radicals.

In the eye, fish oil has been investigated most extensively with regard to age-related macular degeneration (AMD). In large studies, including the Nurses' Health Study and the Health Professionals Follow-up Study, increased dietary fish consumption was associated with a 35% lower risk of age-related macular degeneration. Another prospective, multicenter study found that higher intake of specific types of fat, including vegetable, monounsaturated, and polyunsaturated fats and linoleic acid, rather than total fat intake may be associated with a greater risk for advanced macular degeneration, while diets high in omega-3 fatty acids and fish were inversely associated with risk for macular degeneration when intake of linoleic acid was low.

A combination of DHA, vitamin E and vitamin B were reported to improve both visual field indices and retinal contrast sensitivity in patients with glaucoma. Intramuscular injections of fish oil containing EPA and DHA significantly lowered intraocular pressure in rabbits. Intraperitoneal injection of DHA protected against transient retinal ischemia caused by elevation of introacular pressure, while dietary supplementation of DHA protected against retinal degeneration caused by kainic acid and N-methyl-N-nitrosourea. DHA exerts a protective effect against acute light-induced retinal toxicity.

Alpha-Tocopherol (Vitamin E)

In diabetic rats, treatment with the antioxidant alpha-lipoic acid normalized the amount of leukostasis but not retinal blood flow, while treatment with D-alpha-tocopherol prevented the increases in leukostasis and decreases in retinal blood flow in diabetic rats.

Alpha-tocopherol has been reported to protect against retinal phototoxicity and against ischemic injury of the central nervous system. Alpha-tocopherol has been reported to inhibit human Tenon's capsule fibroblast proliferation and to improve the results of filtering surgery in rabbits. Vitamin E appears to protect against cataract formation and progression in animal models and in humans.

Carnitine

Carnitine, an amino acid derivative found in high energy demanding tissues (skeletal muscles, myocardium, liver), is essential for the intermediary metabolism of fatty acids. It plays an important role in those tissues of the eye, such as the ciliary body, where muscle cells are present and may represent an important energy reserve.

Carnitine prevents glutamate neurotoxicity in primary cultures of cerebellar neurons. It has been reported to prevent retinal injury following ischemia-reperfusion injury. In streptozotocin-diabetic rats, carnitine loss in the lens is an initial and important event and may be related to cataract development. Considerable evidence suggests that mitochondrial dysfunction and oxidative damage may play a role in the pathogenesis of Parkinson's disease and that acetyl-L-carnitine is beneficial in animal models of the disease.

Citicoline and Choline

Choline, as used in the claims of this application, also includes the substance cytidine used individually, or in combination with choline. Citicoline (exogenous CDP-choline) is a nontoxic and well-tolerated drug used in pharmacotherapy of brain insufficiency and some other neurological disorders, such as stroke, brain trauma, and Parkinson's disease. When administered, it undergoes rapid transformation to cytidine and choline, which are believed to enter brain cells separately and provide neuroprotection by enhancing phosphatydylcholine synthesis. A similar effect may be expected to occur in glaucomatous retinal ganglion cells, but the precise effect of citicoline on damaged retinal ganglion cells remains to be explained. In RGC tissue culture, citicoline reduced apoptosis and increased the number of regenerating neurites. Citicoline may induce an improvement of the retinal and visual pathway functions in patients with glaucoma, in whom treatment with citicoline induced a significant (P<0.01) improvement of visual evoked potential and pattern electroretinography (ERG) parameters.

Coenzyme Q10

Tissues which are highly dependent on oxygen such as muscle, the central and peripheral nervous system, kidney, and insulin-producing pancreatic beta-cell are especially susceptible to defective oxidative phosphorylation, which plays an important role in atherogenesis, in the pathogenesis of Alzheimer's disease, Parkinson's disease, diabetes, and aging. Pretreatment of cultured neuronal cells and astrocytes with coenzyme Q10 inhibited cell death due to glutamate neurotoxicity. It also exhibits anti-apoptotic effects, apparently by stabilizing mitochondrial depolarization. Oral Q10 supplementation is effective in treating cardiomyopathies and in restoring plasma levels reduced by the statin type of cholesterol-lowering drugs. Supplementation with Coenzyme Q10 has been reported to slow the development of Parkinson's disease. Patients with open-angle glaucoma have an increased prevalence of Parkinson's disease.

Coenzyme Q10 is beneficial in animal models of neurodegenerative diseases and has shown promising effects both in clinical trials of Parkinson's disease, Huntington's disease and Friedreich's ataxia.

Dan Shen (Salvia miltiorrhiza)

Salvia miltiorrhiza, also known as Asian red sage or Dan shen, contains salviolonic acid B, a potent water-soluble, polyphenolic antioxidant with anti-inflammatory and anti-atherosclerotic properties isolated from Salvia miltiorrhiza. It has been reported to reduce brain damage in cerebral infarctionand mitochondrial damage in ischemia-reperfusion injury.

Retinal ganglion cell damage in glaucomatous damage was markedly reduced by intravenous treatment with S. miltiorrhiza. It has been claimed in one report to stabilize the visual field in patients with glaucoma. Data demonstrate that it inhibits TNF-α-induced activation of NFκβ and in the rabbit model of glaucoma, protects against retinal ganglion cell loss. NMDA receptor antagonist activity may underlie its neuroprotective effects.

Folic Acid

Mild hyperhomocysteinemia is an independent risk factor for premature vascular disease, myocardial infarction, and stroke. Significantly elevated homocysteine levels were also found in patients with Alzheimer's disease as well as in patients with vascular dementia. Homocysteine can induce alterations in extracellular matrix and neuronal cell death that are characteristic findings in glaucoma. Folate supplementation reduces hyperhomocysteinemia, which has been associated with Alzheimer's disease, cardiovascular disease, and glaucoma.

Culturing embryonic cortical neurons and differentiated SH-SY-5Y human neuroblastoma cells in folate-free medium induced neurodegenerative changes characteristic of those observed in Alzheimer's disease. A significant increase in homocysteine was detected following folate deprivation, which decreased the reduced form of glutathione, indicating a depletion of oxidative buffering capacity. A recent study demonstrated that folic acid (400 μg) associated with vitamin B6 and B12 can reduce homocysteine levels by 30%.

Exfoliation syndrome (XFS) is the most common recognizable cause of open-angle glaucoma overall worldwide. XFS is correlated positively with a history of hypertension, angina, myocardial infarction or stroke, suggestive of vascular effects of the disease. XFS has been found in patients with Alzheimer's disease.

Plasma homocysteine levels are elevated in patients with XFS both with and without glaucoma when compared to controls with no ocular disease and to patients with normal-tension glaucoma. Both XFS and hyperhomocysteinemia share common associations with various disorders. Hyperhomocysteinemia might be a modifiable risk factor for XFS. Homocysteine levels and the frequency of heterozygous methylenetetrahydrofolate reductase (MTHFR) C677T mutation are also increased in primary open-angle glaucoma.

A strong protective influence on cortical cataract, from use of folate or vitamin B 12 supplements was found in the Blue Mountains Eye Study.

Ginkgo biloba Extract (GBE)

GBE contains over 60 known bioactive compounds. The standardized extract used most widely in clinical research, EGb 761 (Dr Willmar Schwabe GmbH & Co, Karlsruhe, Germany), contains 24% ginkgo flavone glycosides (flavonoids), 6% terpene lactones (ginkgolides and bilobalide), approximately 7% proanthocyanidines, and other, uncharacterized compounds. In the United States, it is freely available as a nutritional supplement. GBE has been claimed effective in a variety of disorders associated with aging, including cerebrovascular disease, peripheral vascular disease, dementia, tinnitus, bronchoconstriction, and sexual dysfunction. GBE appears to have many properties applicable to the treatment of non-IOP-dependent risk factors for glaucomatous damage (see Ritch, “Potential Role for Ginkgo biloba extract in the treatment of glaucoma” Medical Hypotheses 2000; 54:221-235, the contents of which are hereby incorporated by reference herein).

GBE exerts significant protective effects against free radical damage and lipid peroxidation in various tissues and experimental systems. Its antioxidant potential is comparable to water soluble antioxidants such as ascorbic acid and glutathione and lipid soluble ones such as alpha-tocopherol and retinol acetate. GBE preserves mitochondrial metabolism and adenosine triphosphate (ATP) production in various tissues and partially prevents morphologic changes and indices of oxidative damage associated with mitochondrial aging. It can scavenge nitric oxide and possibly inhibit its production.

Substantial experimental evidence exists to support the view that GBE has neuroprotective properties in conditions such as hypoxia/ischemia, seizure activity, cerebral edema, and peripheral nerve damage. GBE can reduce glutamate-induced elevation of calcium concentrations and can reduce oxidative metabolism in both resting and calcium-loaded neurons. Neurons in tissue culture are protected from a variety of toxic insults by GBE. GBE inhibits apoptosis.

GBE improves both peripheral and cerebral blood flow. It has been reported to protect myocardium against hypoxia and ischemia-reperfusion injury. There is convincing evidence for functional improvement in patients with Alzheimer's-type and multi-infarct dementias.

In the eye, GBE may have a protective effect against the progression of diabetic retinopathy and reduces ischemia-reperfusion injury in rat retina. GBE protects retinal photoreceptors against light-induced damage. Chloroquine-induced ERG changes were prevented by simultaneous treatment with GBE. In a rat model of central retinal artery occlusion, GBE reduced edema and necrosis and blocked the reduction in b-wave amplitude.

GBE has been reported to improve automated visual field indices. In one clinical cross-over study of low-dose, short-term treatment in normal volunteers, GBE increased ophthalmic artery blood flow by a mean of 24%.

Ginseng RB1/RG3

Next to GBE, ginseng, a highly valued herb in the Far East, is the most studied plant compound. Panax ginseng is one of the most widely used herbs in traditional Chinese medicine. The major active components of ginseng are ginsenosides, a diverse group of steroidal saponins, which demonstrate the ability to target a myriad of tissues, producing an array of pharmacological responses. Of greatest interest are the ginsenoside saponins Rb1 and Rg3, which attenuate or inhibit responses that lead to the apoptotic cascade, including glutamate-induced neurotoxicity, calcium influx into cells in the presence of excess glutamate, and lipid peroxidation.

Ginsenosides Rb1 and Rg3 exert significant neuroprotective effects on cultured cortical cells, and apparently act by inhibiting N-methyl-d-aspartate (NMDA) receptor activity. Central infusion of ginsenoside Rb1 in a gerbil model after forebrain ischemia protects hippocampal CA1 neurons against lethal ischemic damage. Ginsenoside Rb1 has been reported to enhance peripheral nerve regeneration in vitro. Ginsenosides suppress tumor necrosis factor-alpha production in vitro and may have potential therapeutic efficacy against TNF-alpha mediated disease.

L-Glutathione

Glutathione is one of the most important antioxidants in the body. Oxidative DNA damage is significantly increased in the trabecular meshwork of glaucoma patients and GSTM1 gene deletion, which has been associated with an increased risk of cancer at various sites and molecular lesions in atherosclerosis, predisposes to more severe damage.

In a study to identify retinal proteins that are the targets of serum autoantibodies in patients with glaucoma, serum antibodies against glutathione S-transferase antigen were recognized in 34 (52%) of 65 patients with glaucoma and 5 (20%) of 25 age-matched controls (P<0.05). These findings indicate that glutathione S-transferase is targeted by the serum antibodies detected in some patients with glaucoma.

A significant association of the glutathione S-transferase M1 polymorphism with primary open-angle glaucoma has been reported. The risk among the GSTM1 positive individuals of developing glaucoma was even higher among smokers. The level of sulfhydryl groups was reported to be significantly lowered in the anterior chamber humor of patients with open-angle glaucoma.

Grape Seed Extract

Grape seed proanthocyanidins have been reported to possess a broad spectrum of pharmacological and medicinal properties against oxidative stress. Grape seed proanthocyanidin extract (GSE) provides excellent protection against free radicals in both in vitro and in vivo models. GSE significantly prevented and postponed development of cataract formation in rats with hereditary cataracts. Improvement in myocardial ischemia-reperfusion injury in vitro has also been reported. Activin, a new generation antioxidant derived from grape seed proanthocyanidins, reduced plasma levels of oxidative stress and adhesion molecules (ICAM-1, VCAM-1 and E-selectin) in patients with systemic sclerosis. Supplementation of a meal with GSE minimizes postprandial oxidative stress by decreasing oxidants and increasing the antioxidant levels in plasma, and, as a consequence, enhancing the resistance to oxidative modification of low density lipoproteins. Grape seed proanthocyanidins have also been reported to have activity against HIV-1 entry into cells.

Green Tea Catechins

Tea contains a number of bioactive chemicals and is particularly rich in catechins, of which epigallocatechin gallate (EGCG) is the most abundant and is an extremely potent antioxidant. Catechins and epicatechins are important constituents in human nutrition. There is a concentration-dependent correlation between these compounds and modulation of cell survival/cell death-related gene pathways in vitro. Catechins reduce mitochondrial damage during ischemia-reperfusion injury. Green tea extract scavenges free radicals and nitric oxide and have been reported to counteract the oxidative insult from cigarette smoke and to retard the progression of cataract. Oxidative alterations of low density lipoproteins, scavenging of oxygen free radicals, and inhibition of glutamate toxicity are properties of catechins.

Lutein and Zeaxanthine

Lutein is one of the most widely found carotenoids distributed in fruits and vegetables frequently consumed. Distribution of lutein among tissues is similar to other carotenoids but, along with zeaxanthin, they are found selectively at the centre of the retina, being usually referred to as macular pigments. Lutein and zeaxanthin may protect the macula and photoreceptor outer segments throughout the retina from oxidative stress and play a role in an antioxidant cascade that safely disarms the energy of reactive oxygen species.

Age-related macular degeneration is the leading cause of blind registration in the developed world. One etiological hypothesis involves oxidation, and the intrinsic vulnerability of the retina to damage via this process. This has prompted interest in the role of antioxidants, particularly the carotenoids lutein and zeaxanthin, in its prevention and treatment. There is ample epidemiological evidence that the amount of macular pigment is inversely associated with the incidence of age-related macular degeneration. Dietary supplementation with lutein and zeaxanthin increase macular pigment density.

Several large, randomized, controlled trials, including the highly publicized Age-Related Eye Disease Study, have examined the role of supplements containing lutein, vitamins C and E, zinc and copper on measures of visual function in people with and without age-related macular disease and have observed a beneficial effect. Amplitudes of focal electroretinograms were improved in patients with ARMD receiving supplementation with lutein, vitamin E, and nicotinamide. Zeaxanthin has been reported to protect retinal photoreceptors from acute light-induced toxicity. Lutein and zeaxanthin may or may not also retard cataract progression.

Methylcobalamin

In patients with glaucoma, studies have shown possible improvement or stabilization in visual field performance with oral B12 supplementation. Methylcobalamin protects cultured retinal ganglion cells against glutamate-induced neurotoxicity.

N-Acetyl-L Cysteine

Apoptosis in retinal microvessels in diabetic retinopathy is associated with an increase in cellular ceramide and diacylglycerol levels. The production of diacylglycerol/ceramide is inhibited by N-acetyl-L-cysteine. Protein carbonylation, a nonenzymatic modification that occurs in conditions of cellular oxidative stress, was inhibited by the N-acetyl-L-cysteine. N-acetyl-L-cysteine increased the neuronal cell survival rate in cultured neurons from embryonic mouse cortex and striatum.

Pycnogenol

Pycnogenol, an extract of French maritime pine bark (Pinus pinaster), primarily composed of procyanidins and phenolic acids, is a potent antioxidant which has strong free radical-scavenging activity against reactive oxygen and nitrogen species. Procyanidins are biopolymers of catechin and epicatechin subunits which are recognized as important constituents in human nutrition. Pretreatment with pycnogenol reduces smoke-induced platelet aggregation. Pycnogenol significantly reduces LDL-cholesterol levels. In patients with chronic venous insufficiency, circumference of the lower legs and symptoms of pain, cramps, night-time swelling, feeling of “heaviness”, and reddening of the skin were reduced.

Glutamate-induced cytotoxicity in HT-4 neuronal cells has been demonstrated to be due to oxidative stress caused by depletion of cellular glutathione (GSH). Extracts of Gingko biloba (EGb 761) and French maritime pine bark (Pycnogenol) were effective inhibitors of this cytotoxicity. Pycnogenol can protect vascular endothelial cells from Aβ-induced injury, suggesting that it may be useful for the prevention and/or treatment of vascular or neurodegenerative diseases associated with Aβ toxicity. PYC not only suppresses the generation of reactive oxygen species, but also attenuates caspase-3 activation and DNA fragmentation, suggesting protection against Aβ-induced apoptosis.

Pycnogenol has also been reported to have angiotensin-converting enzyme (ACE) inhibiting activity, and the ability to enhance the microcirculation by increasing capillary permeability. Pycnogenol inhibits the progression of diabetic retinopathy.

Quercetin

This flavonoid antioxidant, found in Ginkgo biloba extract and in red wine, inhibits release of nitric oxide and tumor necrosis factor alpha, which may be an important factor in the initiation of glaucomatous damage. Quercetin is neuroprotective against oxidative injury in cortical cell cultures, inhibiting lipid peroxidation and scavenging free radicals, and hepatoprotective against ischemia-reperfusion injury when given orally. Apoptosis-promoting substances, including TNF-alpha secreted by activated glial cells after exposure to stress, contribute directly to neuronal cytotoxicity. Quercetin inhibits lipid peroxidation in the mammalian eye and has been reported to slow the progression of selenite-induced cataract in rats.

Resveratrol

Resveratrol is found largely in the skins of red grapes and came to scientific attention as a possible explanation for the low incidence of heart disease among the French, who eat a relatively high-fat diet. Many studies suggest that consuming alcohol (especially red wine) may reduce the incidence of coronary heart disease (CHD). Grape juice, which is not a fermented beverage, is not a significant source of resveratrol.

Several studies have demonstrated that resveratrol is an effective antioxidant. It inhibits lipid peroxidation of low-density lipoprotein (LDL), prevents the cytotoxicity of oxidized LDL, and protects cells against lipid peroxidation. Its antiapoptotic activity has led to the suggestion that resveratrol may make a useful dietary supplement for minimizing oxidative injury in immune-perturbed states and human chronic degenerative diseases.

Levels of Intracellular heme (iron-protoporphyrin IX), a pro-oxidant, increase after stroke, and, in neuronal cell cultures, resveratrol induces heme oxygenase 1, suggesting that increased heme oxygenase activity is a unique pathway by which resveratrol can exert its neuroprotective actions.

Taurine

Taurine is a free amino acid particularly abundant in the retina. Visual dysfunction in both humans and animals results from taurine deficiency, which can be reversed with nutritional supplementation. The distribution of taurine is tightly regulated in the different retinal cell types through the development of the retina. The exact function or functions of taurine in the retina are still unresolved. Nevertheless, taurine depletion results in significant retinal lesions, and taurine release and uptake has been found to employ distinct regulatory mechanisms in the retina.

Taurine supplementation in diabetic rats significantly decreases lipid peroxidation and preserves ATPase activity. Taurine protected against low level radiation-associated protein leakage. In studies of neuritic outgrowth from post-crush goldfish retinal explants, the highest neuritic outgrowth was observed in the presence of fetal calf serum, in which condition the amino acid taurine increased length and density of neurites. Treatment with taurine, diltiazem, and vitamin E, had a beneficial effect of decreasing the rate of visual field loss in patients with retinitis pigmentosa, likely through a protective action from free radical reactions in affected photoreceptors.

Bilberry

Compounds from bilberry extracts and preparations have been touted to provide a benefit to night visual acuity and/or night contrast sensitivity, and were supposedly used by RAF pilots during World War II. Although research on healthy, non-visually impaired, patients show no benefit for night visual acuity or night contrast sensitivity, there is a total absence of research on patients with pathologically impaired night vision—or night blindness.

Hawthorne (Cratageous spp.)

Hawthorne preparations have been used to treat angina and arrhythmias, and congestive heart failure (CHF) around the world, particularly in Europe and Asia (see Stephen Foster, “Hawthorn” [online] copyright 2000. Retrieved from the internet:<URL:http://www.stevenfoster.com/education/monograph/hawthorn.html). Patients with CHF taking hawthorne preparations have shown improved exercise tolerance, relative to a placebo control group. In addition, there are reports that hawthorne may have anti-ischemic and lipid-lowering potential.

Arctigenin

Arctigenin is a lignan isolated from various plant species, including Torreya nucifera and Arctium lappa, that may regulate immune responses in activated macrophages and lymphocytes. It is known to inhibit tumor necrosis factor-alpha (TNF-α) and nitric oxide (NO) production, and to protect cultured neurons from glutamate toxicity. It is also known to be have anti-HIV-1 activity, presumably by its ability to inhibit the HIV-1 enzyme integrase, and it is also an inhibitor of DNA topoisomerase II.

Scrophularia

Compounds from Scrophularia extracts and preparations, particularly iridoid glycosides and E-p-methoxycinnamic acid have been found to inhibit calcium influx into cells. These preparations also are known to inhibit glutamate-induced neurotoxicity in cultured rat cortical neurons, so are good candidates to include in formulations for treating and preventing glaucoma using neuroprotective strategies.

Forskolin

Forskolin extract (from Coleus forskohlii) and its uses have been widely popularized and publicized on the internet. Claims to its medicinal benefits have been investigated and such extracts have been found to reduce aqueous secretion. One human single-dose study has suggested that topical application lowers IOP, and so extracts from this plant are promising in the treatment of glaucoma and related conditions, although there is some suggestion that continued use may result in tachyphylaxis.

S-Allylmercaptocysteine

S-Allylmercaptocysteine has been reported to lower IOP in rabbits by acting to increase atrial natriuretic peptide (ANP) (see J Ocular Pharm Ther 1999; 15:9-17). Similar results are expected to occur in humans.

Glucosamine

A report by Meininger et al. (Biochem Biophys Res Commun 2000; 279:234) indicates that glucosamine inhibits inducible nitric oxide synthase (iNOS). Since inhibition of NO syntase is associated with neuroprotection in non-glaucoma conditions, it is reasonable to infer that formulations containing inhibitors of iNOS may also act to provide neuroprotection and aid in the treatment of glaucoma.

Embodiments of the present invention utilize one or more of the aforementioned compounds in a neuroprotective formulation to lower the non-IOP risk factors of glaucoma. Some embodiments of the invention may act to prevent or limit the progression of glaucoma by strengthening the resistance of nerve cells to damage from glaucoma. The formulations may combine specific quantities of one or more of the compounds in a dosage formulation for use by a patient. The specific quantity utilized may be any amount that provides convenience for administration of the dosage formulation to a patient. For example, a dosage formulation may be in the form of one or more easily swallowed pills, wherein the one or more pills is administered to a patient on a scheduled basis; such pills may contain an identical composition and quantity of each component in each pill, or pills may vary in composition and/or quantity of components. Dosage formulations may also be in the form of liquids, semi-solids, or other forms which are readily formed by those skilled in the art.

In a specific embodiment of the invention, a neuroprotective formulation includes the compounds ginkgo biloba extract, resveratrol, and pycnogenol. In a second specific embodiment of the invention, a neuroprotective formulation includes carnitine, methylcobalamin, and coenzyme Q10. In a third specific embodiment of the invention, a neuroprotective formulation includes ginseng extract and salvia militiorrhiza. A fourth specific embodiment of the invention is directed toward a neuroprotective formulation that includes ginko biloba extract and salvia militiorrhiza. A fifth specific embodiment of the invention is directed toward a neuroprotective formulation that includes camitine and folic acid. In a sixth specific embodiment, a neuroprotective formulation includes pycnogenol, alpha-lipoic acid, and fish oil. Other specific embodiments of the invention are directed toward neuroprotective formulations that include one or more components of the previously described six specific embodiments.

In another specific embodiment of the invention, a neuroprotective formulation includes the following compounds in the corresponding amounts corresponding (when listed):

| | | |
|---|---|---|
| Alpha-lipoic acid | 100 | mg |
| Fish oil | 500 | mg |
| Alpha-tocopherol | 200 | Units |
| Carnitine | 250 | mg |
| Choline | 100 | mg |
| Coenzyme Q10 | 50 | mg |
| Folic acid | 400 | mcg |
| GBE | 90 | mg |
| 1-Glutathione | 250 | mg |
| Grape seed extract | 100 | mg |
| Green tea catechins | 100 | mg |
| Lutein | 20 | mg |
| Methylcobalamin | 500 | mcg |
| N-acetyl-L cysteine | 200 | mg |
| Pycnogenol | 100 | mg |
| Quercetin | 200 | mg |
| Resveratrol | 20 | mg |
| Taurine | 200 | mg |
| Zeaxanthine | 2 | mg |
| Copper | 2 | mg |
| Magnesium | 200 | mg |
| Selenium | 10 | mg |
| Zinc | 25 | mg |
| Ginseng Rb1/Rg3 | | |
| Panax ginseng C. A. Meyer (Araliaceae) | | |
| Salvia miltiorrhiza | | |

Alternate embodiments of the invention may utilize one or more of the above-listed compounds in quantities that differ from those stated in the list.

Embodiments of the present invention may further include vitamins, minerals, and other components which may promote the general health of a patient. Non-limiting examples of such components include one or more of copper, magnesium, selenium, zinc and vitamin A. The amounts of each component may vary, and may correspond to a daily allowance as established by a governmental body or scientific or health panel.

Other embodiments of the invention may utilize a neuroprotective formulation in a method of reducing the risk of one or more non-intraocular pressure-dependent factors of glaucoma. The method includes the steps of: providing the neuroprotective formulation; and administering the formulation to a patient. Neuroprotective formulations for use with such a method include, but are not limited to, the neuroprotective formulations described herein, and formulations utilizing one of more of the compounds described herein. Such formulations may be constructed is a dosage formulation for convenient administration to a patient. Administering such formulations may include one or more of the functions of administering a solid formulation, injecting the formulation, or administering the formulation orally in a liquid form. Alternatively, the method may prevent the progression of glaucoma by strengthening the resistance of damage to nerve cells.

It is understood that the present invention is not to be limited by the embodiments of the invention described herein. Indeed, those skilled in the art will readily understand that various modifications and embodiments of the invention may be made and practiced without departing from the scope of the invention.

### REFERENCES

1. Goralska M, Dackor R, Holley B, McGahan M C. Alpha lipoic acid changes iron uptake and storage in lens epithelial cells. Exp Eye Res 2003;76:241-8.
2. Borenshtein D, Ofri R, Werman M, et al. Cataract development in diabetic sand rats treated with alpha-lipoic acid and its gamma-linolenic acid conjugate. Diabetes Metab Res Rev 2001;17:44-50.
3. Maitra I, Serbinova E, Tritschler H J, Packer L. Stereospecific effects of R-lipoic acid on buthionine sulfoximine-induced cataract formation in newborn rats. Biochem Biophys Res Commun 1996;221:422-9.
4. Abiko T, Abiko A, Clermont A C, et al. Characterization of retinal leukostasis and hemodynamics in insulin resistance and diabetes: role of oxidants and protein kinase-C activation. Diabetes 2003;52:829-37.
5. Rotstein N P, Politi L E, German O L, Girotti R. Protective effect of docosahexaenoic acid on oxidative stress-induced apoptosis of retina photoreceptors. Invest Ophthalmol Vis Sci 2003;44:2252-9.
6. Miyauchi O, Mizota A, Adachi-Usami E, Nishikawa M. Protective effect of docosahexaenoic acid against retinal ischemic injury: an electroretinographic study. Ophthalmic Res 2001;33:191-195.
7. Mizota A, Sato E, Taniai M, et al. Protective effects of dietary docosahexaenoic acid against kainate-induced retinal degeneration in rats. Invest Ophthalmol Vis Sci 2001;42:216-221.
8. Murayama K, Yoneya S, Miyauchi O, et al. Fish Oil (Polyunsaturated Fatty Acid) Prevents Ischemic-induced Injury in the Mammalian Retina. Exp Eye Res 2002;74:671-676.
9. Cho E, Hung S, Willett W C, et al. Prospective study of dietary fat and the risk of age-related macular degeneration. Am J Clin Nutr 2001;73:209-218.
10. Smith W, Mitchell P, Leeder S R. Dietary fat and fish intake and age-related maculopathy. Arch Ophthalmol 2000;118:401-4.
11. Seddon J M, Rosner B, Sperduto R D, et al. Dietary fat and risk for advanced age-related macular degeneration. Arch Ophthalmol 2001;119:1191-1199.
12. Cellini M, Caramazza N, Mangiafico P, et al. Fatty acid use in glaucomatous optic neuropathy treatment. Acta Ophthalmol Scand 1998;Suppl 227:41-42.
13. Mancino M, Ohia E, Kulkarni P. A comparative study between cod liver oil and liquid lard intake on IOP in rabbits. Prostaglandins Leukot Essent Fatty Acids 1992;45:239-243.
14. Moriguchi K, Yuri T, Yoshizawa K, et al. Dietary docosahexaenoic acid protects against N-methyl-N-nitrosourea-induced retinal degeneration in rats. Exp Eye Res 2003;77:167-73.
15. Reme C E, Malnoe A, Jung H H, et al. Effect of dietary fish oil on acute light-induced photoreceptor damage in the rat retina. Invest Ophthalmol Vis Sci 1994;35:78-90.
16. Aonuma H, Koide K, Masuda K, Watanabe I. Retinal light damage: Protective effect of alpha-tocopherol. Jpn J Ophthalmol 1997;41:160-167.
17. Tagami M, Yamagata K, Ikeda K, et al. Vitamin E prevents apoptosis in cortical neurons during hypoxia and oxygen reperfusion. Lab Invest 1998;78:1415-1429.
18. Takahashi H, Kosaka N, Nakagawa S. alpha-Tocopherol protects PC12 cells from hyperoxia-induced apoptosis. J Neurosci Res 1998;52:184-191.
19. Van der Worp H B, Bar P R, Kappelle L J, de Wildt D J. Dietary vitamin E levels affect outcome of permanent focal cerebral ischemia in rats. Stroke 1998;29:1002-1005.
20. Haas A L, Boscoboinik D, Mojon D S, et al. Vitamin E inhibits proliferation of human Tenon's capsule fibroblasts in vitro. Ophthalmic Res 1996;28:171-175.
21. Pinilla I, Larrosa J M, Polo V, Honrubia F M. Alpha-tocopherol derivatives in an experimental model of filtering surgery. Ophthalmic Res 1999;31:440-5.
22. Kuzniarz M, Mitchell P, Cumming R G, Flood V M. Use of vitamin supplements and cataract: the Blue Mountains Eye Study. Am J Ophthalmol 2001;132:19-26.
23. Kojima M, Shui Y B, Murano H, Sasaki K. Inhibition of steroid-induced cataract in rat eyes by administration of vitamin-E ophthalmic solution. Ophthalmic Res 1996;28(Suppl 2):64-7.
24. Nagata M, Kojima M, Sasaki K. Effect of vitamin E eye drops on naphthalene-induced cataract in rats. J Ocular Pharmacol Ther 1999;15:345-350.
25. Rouhiainen P, Rouhiainen H, Salonen J T. Association between low plasma vitamin E concentration and progression of early cortical lens opacities. Am J Epidemiol 1996; 144:496-500.
26. Pessotto P, Valeri P, Arrigoni-Martelli E. The presence of L-carnitine in ocular tissues of the rabbit. J Ocul Pharmacol 1994;10:643-51.
27. Llansola M, Erceg S, Hernandez-Viadel M, Felipo V. Prevention of ammonia and glutamate neurotoxicity by carnitine: molecular mechanisms. Metab Brain Dis 2002;17:389-397.
28. Kocer I, Kulacoglu D, Altuntas I, et al. Protection of the retina from ischenia-reperfusion injury by L-carnitine in guinea pigs. Eur J Ophthalmol 2003;13:80-85.
29. Beal M F. Bioenergetic approaches for neuroprotection in Parkinson's disease. Ann Neurol 2003;53 Suppl 3:S39-47.
30. Grieb P, Rejdak R. Pharmacodynamics of citicoline relevant to the treatment of glaucoma. J Neurosci Res 2002;67:143-8.
31. Oshitari T, Fujimoto N, Adachi-Usami E. Citicoline has a protective effect on damaged retinal ganglion cells in mouse culture retina. Neuroreport 2002;13:2109-11.
32. Parisi V, Manni G, Colacino G, Bucci M G. Cytidine-5′-diphosphocholine (citicoline) improves retinal and cortical responses in patients with glaucoma. Ophthalmology 1999;106:1126-34.
33. Rejdak R, Toczolowski J, Kurkowski J, et al. Oral citicoline treatment improves visual pathway function in glaucoma. Med Sci Monit 2003;9:PI24-8.
34. Fosslien E. Mitochondrial medicine—molecular pathology of defective oxidative phosphorylation. Ann Clin Lab Sci 2001;31:25-67.
35. Sandhu J K, Pandey S, Ribecco-Lutkiewicz M, et al. Molecular mechanisms of glutamate neurotoxicity in mixed cultures of NT2-derived neurons and astrocytes: Protective effects of coenzyme Q10. J Neurosci Res 2003;72:691-703.
36. Papucci L, Schiavone N, Witort E, et al. Coenzyme Q10 prevents apoptosis by inhibiting mitochondrial depolarization independently of its free radical-scavenging property. J Biol Chem 2003;.
37. Shults C W, Oakes D, Kieburtz K, et al. Effects of coenzyme Q(10) in early Parkinson disease-Evidence of slowing of the functional decline. Arch Neurol 2002;59:1541-1552.
38. Bayer A U, Keller O N, Ferrari F, Maag K P. Association of glaucoma with neurodegenerative diseases with apoptotic cell death: Alzheimer's disease and Parkinson's disease. Am J Ophthalmol 2002;133:135-137.
39. Chen Y H, Lin S J, Ku H H, et al. Salvianolic acid B attenuates VCAM-1 and ICAM-1 expression in TNF-alpha-treated human aortic endothelial cells. J Cell Biochem 2001;82:512-521.
40. Wu Y J, Hong C Y, Lin S J, et al. Increase of vitamin E content in LDL and reduction of atherosclerosis in cholesterol-fed rabbits by a water-soluble antioxidant-rich fraction of Salvia miltiorrhiza. Arterioscler Thromb Vasc Biol 1998;18:481-486.
41. Min L Q, Dang L Y, Ma W Y. [Clinical study on effect and therapeutical mechanism of composite Salvia injection on acute cerebral infarction]. Zhongguo Zhong Xi Yi Jie He Za Zhi 2002;22:353-5.
42. Lam BY, Lo AC, Sun X, et al. Neuroprotective effects of tanshinones in transient focal cerebral ischemia in mice. Phytomedicine 2003;10:286-91.
43. Zhang W H, Wang J S, Zhou Y, Li J Y. Gadolinium chloride and salvia miltiorrhiza compound ameliorate reperfusion injury in hepatocellular mitochondria. World J Gastroenterol 2003;9:2040-4.
44. Zhu M D, Cai F Y. Evidence of compromised circulation in the pathogenesis of optic nerve damage in chronic glaucomatous rabbit. Chin Med J 1993;106:922-927.
45. Wu Z Z, Jiang Y Q, Yi S M, Xia M T. Radix salviae miltiorrhizae in middle and late-stage glaucoma. Chinese Med J 1983;96:445-447.
46. Sun X, Chan L N, Gong X, Sucher N J. N-methyl-D-aspartate receptor antagonist activity in traditional Chinese stroke medicines. Neurosignals 2003;12:31-38.
47. Clarke R, Daily L, Robinson K, et al. Hypermocysteinemia: An independent risk factor for vascular disease. N Engl J Med 1991;324:1149-1155.
48. Stampfer M J, Malinow M R, Willet W C, et al. A prospective study of plasma homocysteine and risk of myocardial infarction in US physicians. JAMA 1992;268:877-881.
49. Perry I J, Refsum H, Morris R W, et al. Prospective study of serum total homocysteine concentration and risk of stroke in middle-aged British me. Lancet 1995;346:1395-1398.
50. Leblhuber F, Walli J, Artner-Dworzak E, et al. Hyperhomocysteinemia in dementia. J Neural Transm 2000;107:1469-1474.
51. Ho P I, Ashline D, Dhitavat S, et al. Folate deprivation induces neurodegeneration: roles of oxidative stress and increased homocysteine. Neurobiol Dis 2003;14:32-42.
52. Lobo A, Naso A, Arheart K, et al. Reduction of homocysteine levels in coronary artery disease by low-dose folic acid combined with vitamins B6 and B 12. Am J Cardiol 1999;83:821-825.
53. Ritch R. Exfoliation syndrome: The most common identifiable cause of open-angle glaucoma. J Glaucoma 1994;3:176-178.
54. Mitchell P, Wang J J, Smith W. Association of pseudoexfoliation with increased vascular risk. Am J Ophthalmol 1997;124:685-687.
55. Linnér E, Popovic V, Gottfries C G, et al. The exfoliation syndrome in cognitive impairment of cerebrovascular or Alzheimer's type. Acta Ophthalmol Scand 2001;79:283-285.
56. Vessani R M, Liebmann J M, Jofe M, Ritch R. Plasma homocysteine is elevated in patients with exfoliation syndrome. Am J Ophthalmol 2003;136:41-46.
57. Leibovitch I, Kurtz S, Shemesh G, et al. Hyperhomocystinemia in pseudoexfoliation glaucoma. J Glaucoma 2003;12:36-39.
58. Bleich S, Jünemann A, von Ahsen N, et al. Homocysteine and risk of open-angle glaucoma. J Neural Transmission 2002;109:1499-1504.
59. Jünemann AG, von Ahsen B, Kornhuber H, et al. MTHFR C677T Polymorphism Is a Genetic Risk Factor for Primary Open-Angle Glaucoma. ARVO abstracts 2003;.
60. Ritch R. Potential role for Ginkgo biloba extract in the treatment of glaucoma. Medical Hypotheses 2000;54:221-235.
61. De Feudis FV. Ginkgo biloba Extract (EGb 761): Pharmacological activities and clinical applications.Paris: Elsevier, 1991
62. Köse K, Dogan P. Lipoperoxidation induced by hydrogen peroxide in human erythrocyte membranes. 2. Comparison of the antioxidant effect of Ginkgo biloba extract (EGb 761) with those of water-soluble and lipid-soluble antioxidants. J Int Med Res 1995;23:9-18.
63. Janssens D, Delaive E, Remacle J, Michiels C. Protection by bilobalide of the ischaemia-induced alterations of the mitochondrial respiratory activity. Fundam Clin Pharmacol 2000;14:193-201.
64. Pierre S, Jamme I, Robert K, et al. GBE (EGb 761) protects Na,K-ATPase isoenzymes during cerebral ischemia. Cell Mol Biol 2002;48:671-680.
65. Sastre J, Lloret A, Borras C, et al. GBE EGb 761 protects against mitochondrial aging in the brain and in the liver. Cell Mol Biol 2002;48:685-692.
66. Marcocci L, Maguire J J, Droy-Lefaix M T, Packer L. The nitric oxide-scavenging properties of Ginkgo biloba extract (EGb 761). Biochem Biophys Res Commun 1994;201:748-755.
67. Kobuchi H, Droy-Lefaix M T, Christen Y, Packer L. Ginkgo biloba extract (EGb 761): Inhibitory effect on nitric oxide production in the macrophage cell line RAW 264.7. Biochem Pharmacol 1997;53:897-904.
68. Smith P F, Maclennan K, Darlington C L. The neuroprotective properties of the Ginkgo biloba leaf: a review of the possible relationshiop to platelet-activating factor (PAF). J Ethnopharmacol 1996;50:131-139.
69. Ahlemeyer B, Krieglstein J. Pharmacological studies supporting the therapeutic use of Ginkgo biloba extract for Alzheimer's disease. Pharmacopsychiatry 2003;36 Suppl 1:S8-14.
70. Zhu L, Wu J, Liao H, et al. Antagonistic effects of extract from leaves of Ginkgo biloba on glutamate neurotoxicity. Acta Pharmacol Sinica 1997;18:344-347.
71. Oyama Y, Fuchs P A, Katayama N, Noda K. Myricetin and quercetin, the flavonoid constituents of Ginkgo biloba extract, greatly reduce oxidative metabolism in both resting and Ca(2+)-loaded brain neurons. Brain Res 1994;635:125-129.
72. Bastianetto S, Quirion R. EGb 761 is a neuroprotective agent against 13-amyloid toxicity. Cell Mol Biol 2002;48:693-698.
73. Soulié C, Nicole A, Christen Y, Ceballos-Picot I. The GBE EGb 761 increases viability of hnt human neurons in culture and affects the expression of genes implicated in the stress response. Cell Mol Biol 2002;48:641-646.
74. Guidetti C, Paracchini S, Lucchini S, et al. Prevention of neuronal cell damage induced by oxidative stress in vitro: effect of different Ginkgo biloba extracts. J Pharmacy Pharmacol 2001;53:387-392.
75. Zhou U, Zhu X Z. Reactive oxygen species-induced apoptosis in PC12 cells and protective effect of bilobalide. J Pharmacol Exp Ther 2000;293:982-988.
76. Ahlemeyer B, Mowes A, Krieglstein J. Inhibition of serum deprivation- and staurosporine-induced neuronal apoptosis by Ginkgo biloba extract and some of its constituents. Eur J Pharmacol 1999;367:423-430.
77. Haramaki N, Aggarwal S, Kawabata T, et al. Effects of natural antioxidant Ginkgo biloba extract (EGb 761). on myocardial ischemia-reperfusion injury. Free Radic Biol Med 1994;16:789-794.
78. Punkt K, Welt K, Schaffranietz L. Changes of enzyme activities in the rat myocardium caused by experimental hypoxia with and without ginkgo biloba extract EGb 761 pretreatment. A cytophotometrical study. Acta Histochem 1995;97:67-79.
79. Hofferberth B. The efficacy of EGb 761 in patients with senile dementia of the Alzheimer type. A double-blind, placebo-controlled study on different levels of investigation. Human Psychopharmacol 1994;9:215-222.
80. Le Bars P L, Katz M M, Berman N, et al. A Placebo-controlled, double-blind, randomized trial of an extract of Ginkgo biloba for dementia. JAMA 1997;278:1327-1332.
81. Droy-Lefaix M T, Szabo-Tosaki M E, Doly M N. Free radical scavenger properties of EGb 761 on functional disorders induced by experimental diabetic retinopathy. In: Cutler RG, Packe L, Bertram J, Mori A, ed. Oxidative stress and aging. Basel: Birkhauser Verlag, 1996:277-286.
82. Szabo M E, Droy-Lefaix M T, Doly M, Braquet P. Modification of ischemia/reperfusion-induced ion shifts (Na+, K+, Ca2+ and Mg2+ by free radical scavengers in the rat retina. Ophthalmic Res 1993;25:1.
83. Ranchon I, Gorrand J M, Cluzel J, et al. Functional protection of photoreceptors from light-induced damage by dimethylthiourea and Ginkgo biloba extract. Invest Ophthalmol Vis Sci 1999;40:1191-1199.
84. Meyniel G, Doly M, Millerin M, Braquet P. Involvement of PAF (Platelet-Activating Factor) in chloroquine-induced retinopathy. C R Acad Sci III 1992;314:61-5.
85. Droy-Lefaix M T, Szabo M E, Doly M N. Ischaemia and reperfusion-induced injury in the retina obtained form normotensive and spontaneously hypertensive rats: effects of free radical scavengers. Int J Tissue React 1993;15:85-91.
86. Raabe A, Raabe M, Ihm P. Therapeutic follow-up using automatic perimetry in chronic cerebroretinal ischemia in elderly patients. Prospective double-blind study with graduated dose Ginkgo biloba treatment. Klin Monatsbl Augenheilkd 1991;199:432-438.
87. Quaranta L, Bettelli S, Uva M G, et al. Effect of Ginkgo biloba extract on pre-existing visual field damage in normal tension glaucoma. Ophthalmology 2003;110:359-364.
88. Chung H S, Harris A, Kristinsson J K, et al. Ginkgo biloba extract increases ocular blood flow velocity. J Ocular Pharmacol Therap 1999;15:233-240.
89. Attele A S, Wu J A, Yuan C S. Ginseng pharmacology: multiple constituents and multiple actions. Biochem Pharmacol 1999;58:1685-1693.
90. Kim Y C, Kim S R, Markelonis G J, Oh T H. Ginsenosides Rb1 and Rg3 protect cultured rat cortical cells from glutamate-induced neurodegeneration. J Neurosci Res 1998;53:426-432.
91. Kim S, Ahn K, Oh T H, et al. Inhibitory effect of ginsenosides on NMDA receptor-mediated signals in rat hippocampal neurons. Biochem Biophys Res Commun 2002;296:247-254.
92. Lim J H, Wen T C, S. M, et al. Protection of ischemic hippocampal neurons by ginsenoside Rb1, a main ingredient of ginseng root. Neurosci Res 1997;:191-200.
93. Chen Y S, Wu C H, Yao C H, Chen C T. Ginsenoside Rb1 enhances peripheral nerve regeneration across wide gaps in silicone rubber chambers. Int J Artif Organs 2002;25:1103-1108.
94. Cho J Y, Yoo E S, Baik K U, et al. In vitro inhibitory effect of protopanaxadiol ginsenosides on tumor necrosis factor (TNF)-alpha production and its modulation by known TNF-alpha antagonists. Planta Med 2001;67:213-218.
95. Izzotti A, Sacca S C, Cartiglia C, De Flora S. Oxidative deoxyribonucleic acid damage in the eyes of glaucoma patients. Am J Med 2003;114:638-46.
96. Yang J, Tezel G, Patil R V, et al. Serum autoantibody against glutathione S-transferase in patients with glaucoma. Invest Ophthalmol Vis Sci 2001;42:1273-6.
97. Juronen E, Tasa G, Veromann S, et al. Polymorphic glutathione S-transferase M1 is a risk factor of primary open-angle glaucoma among Estonians. Exp Eye Res 2000;71:447-52.
98. Bunin A I, Filina A A, Erichev V P. A glutathione deficiency in open-angle glaucoma and the approaches to its correction. Vestn Oftalmol 1992;108:13-5.
99. Bagchi D, Bagchi M, Stohs S, et al. Cellular protection with proanthocyanidins derived from grape seeds. Ann NY Acad Sci 2002;957:260-270.
100. Yamakoshi J, Saito M, Kataoka S, Tokutake S. Procyanidin-rich extract from grape seeds prevents cataract formation in hereditary cataractous (ICR/f) rats. J Agric Food Chem 2002;50:4983-4988.
101. Pataki T, Bak I, Kovacs P, et al. Grape seed proanthocyanidins improved cardiac recovery during reperfusion after ischemia in isolated rat hearts. Am J Clin Nutrition 2002;75:894-899.
102. Shao Z H, Becker L B, Vanden Hoek T L, et al. Grape seed proanthocyanidin extract attenuates oxidant injury in cardiomyocytes. Pharmacol Res 2003;47:463-469.
103. Bagchi D, Sen C K, Ray S D, et al. Molecular mechanisms of cardioprotection by a novel grape seed proanthocyanidin extract. Mutat Res 2003;523-524:87-97.
104. Kalin R, Righi A, Del Rosso A, et al. Activin, a grape seed-derived proanthocyanidin extract, reduces plasma levels of oxidative stress and adhesion molecules (ICAM-1, VCAM-1 and E-selectin) in systemic sclerosis. Free Radical Res 2002;36:819-825.
105. Natella F, Belelli F, Gentili V, et al. Grape seed proanthocyanidins prevent plasma postprandial oxidative stress in humans. J Agric Food Chem 2002;50:7720-7725.
106. Nair MP, Kandaswami C, Mahajan S, et al. Grape seed extract proanthocyanidins downregulate HIV-1 entry coreceptors, CCR2b, CCR3 and CCR5 gene expression by normal peripheral blood mononuclear cells. Biol Res 2002;35:421-431.
107. Higdon J V, Frei B. Tea catechins and polyphenols: health effects, metabolism, and antioxidant functions. Crit Rev Food Sci Nutr 2003;43:89-143.
108. Lee S R, Im K J, Suh S I, Jung J G. Protective effect of green tea polyphenol (−)-epigallocatechin gallate and other antioxidants on lipid peroxidation in gerbil brain homogenates. Phytother Res 2003;17:206-209.
109. Weinreb O, Mandel S, Youdim M B. cDNA gene expression profile homology of antioxidants and their antiapoptotic and proapoptotic activities in human neuroblastoma cells. FASEB J 2003;17:935-937.
110. van Jaarsveld H, Kuyl J M, Schulenburg D H, Wild N M. Effect of flavonoids on the outcome of myocardial mitochondrial ischemia/reperfusion injury. Res Commun Mol Pathol Pharmacol 1996;91:65-75.
111. Nakagawa T, Yokozawa T. Direct scavenging of nitric oxide and superoxide by green tea. Food Chem Toxicol 2002;40:1745-1750.
112. Thiagarajan G, Chandani S, Sundari C S, et al. Antioxidant properties of green and black tea, and their potential ability to retard the progression of eye lens cataract. Exp Eye Res 2001;73:393-401.
113. Gupta S K, Halde N, Sivastava S, et al. Green tea (Camellia sinensis) protects against selenite-induced oxidative stress in experimental cataractogenesis. Ophthalmic Res 2002;34:258-263.
114. Kakuda T. Neuroprotective effects of the green tea components theanine and catechins. Biol Pharm Bull 2002;25:1513-1518.
115. Bone R A, Landrum J T, Guerra L H, Ruiz C. Lutein and zeaxanthin dietary supplements raise macular pigment density and serum concentrations of these carotenoids in humans. J Nutr 2003;133:992-8.
116. Age-Related Eye Disease Study Research Group. A randomized, placebo-controlled, clinical trial of high-dose supplementation with vitamins C and E, beta carotene, and zinc for age-related macular degeneration and vision loss. AREDS report No. 8. Arch Ophthalmol 2001;119:1417-1436.
117. Falsini B, Piccardi M, larossi G, et al. Influence of short-term antioxidant supplementation on macular function in age-related maculopathy: a pilot study including electrophysiologic assessment. Ophthalmology 2003;110:51-60.
118. Thomson L R, Toyoda Y, Delori F C, et al. Long term dietary supplementation with zeaxanthin reduces photoreceptor death in light-damaged Japanese quail. Exp Eye Res 2002;75:529-42.
119. Berendschot T T, Broekmans W M, Klopping-Ketelaars I A, et al. Lens aging in relation to nutritional determinants and possible risk factors for age-related cataract. Arch Ophthalmol 2002;120:1732-7.
120. Jacques P F, Chylack L T, Jr., Hankinson S E, et al. Long-term nutrient intake and early age-related nuclear lens opacities. Arch Ophthalmol 2001;119:1009-19.
121. Age-Related Eye Disease Study Research Group T. A randomized, placebo-controlled, clinical trial of high-dose supplementation with vitamins C and E and beta carotene, and zinc for age-related cataract and vision loss. AREDS report No. 9. Arch Ophthalmol 2001;119:1439-1452.
122. Azumi I, Kosaki H, Nakatani H. Effects of metcobalamin (Methylcobal) on the visual field of chronic glaucoma—a multicenter open study. Folia Ophthalmol Jpn 1983;34:873-878.
123. Yamazaki Y, Hayamizu F, Tanaka C. Effects of long-term methylcobalamin treatment on the progression of visual field defects in normal-tension glaucoma. Curr Therap Res 2000;61:443-451.
124. Kikuchi M, Kashii S, Honda Y, et al. Protective effects of methylcobalamin, a vitamin B12 analog, against glutamate-induced neurotoxicity in retinal cell culture. Invest Ophthalmol Vis Sci 1997;38:848-854.
125. Denis U, Lecomte M, Paget C, et al. Advanced glycation end-products induce apoptosis of bovine retinal pericytes in culture: involvement of diacylglycerol/ceramide production and oxidative stress induction. Free Radic Biol Med 2002;33:236-47.
126. England K, O'Driscoll C, Cotter T G. Carbonylation of glycolytic proteins is a key response to drug-induced oxidative stress and apoptosis. Cell Death Differ 2003;.
127. Hori K, Katayama M, Sato N, et al. Neuroprotection by glial cells through adult T cell leukemia-derived factor/human thioredoxin (ADF/TRX). Brain Res 1994;652:304-10.
128. Rohdewald P. A review of the French maritime pine bark extract (Pycnogenol), a herbal medication with a diverse clinical pharmacology. Int J Clin Pharmacol Ther 2002;40:158-68.
129. Araghi-Niknam M, Hosseini S, Larson D, et al. Pine bark extract reduces platelet aggregation. Integrative Med 2000;2:73-77.
130. Devaraj S, Vega-Lopez S, Kaul N S, F., et al. Supplementation with a pine bark extract rich in polyphenols increases plasma antioxidant capacity and alters the plasma lipoprotein profile. Lipids 2002;37:931-934.
131. Koch R. Comparative study of Venostasin and Pycnogenol in chronic venous insufficiency. Phytother Res 2002;16 Suppl 1:S1-5.
132. Kobayashi M S, Han D, Packer L. Antioxidants and herbal extracts protect HT-4 neuronal cells against glutamate-induced cytotoxicity. Free Radic Res 2000;32:115-124.
133. Liu F, Lau B H, Peng Q, Shah V. Pycnogenol protects vascular endothelial cells from beta-amyloid-induced injury. Biol Pharm Bull 2000;23:735-737.
134. Peng Q L, Buz'Zard A R, Lau B H. Pycnogenol((R)) protects neurons from amyloid-beta peptide-induced apoptosis. Brain Res Mol Brain Res 2002;104:55-65.
135. Packer L, Rimbach G, Virgili F. Antioxidant activity and biologic properties of a procyanidin-rich extract from pine (Pinus maritima) bark, pycnogenol. Free Radic Biol Med 1999;27:704-24.
136. Schonlau F, Rohdewald P. Pycnogenol for diabetic retinopathy. A review. Int Ophthalmol 2001;24:161-171.
137. Wadsworth T L, Koop D. Effects of Ginkgo biloba extract (EGb 761) and quercetin on lipopolysaccharide-induced release of nitric oxide. Chem-Biol Interact 2001;137:43-58.
138. Wadsworth T L, McDonald T L, D. R. K. Effects of Ginkgo biloba extract (EGb 761) and quercetin on lipopolysaccharide-induced signaling pathways involved in the release of tumor necrosis factor-alpha. Biochem Pharmacol 2001;62:963-974.
139. Dok-Go H, Lee K H, Kim H J, et al. Neuroprotective effects of antioxidative flavonoids, quercetin, (+)-dihydroquercetin and quercetin 3-methyl ether, isolated from Opuntia ficus-indica var. saboten. Brain Res 2003;965:130-136.
140. Su J F, Guo C J, Wei J Y, et al. Protection against hepatic ischemia-reperfusion injury in rats by oral pretreatment with quercetin. Biomed Environ Sci 2003;16:1-8.
141. Tezel G, Wax M. Increased production of tumor necrosis factor-alpha by glial cells exposed to simulated ischemia or elevated hydrostatic pressure induces apoptosis in cocultured retinal ganglion cells. J Neurosci 2000;20:8693-8700.
142. Ueda T, Ueda T, Armstrong D. Preventive effect of natural and synthetic antioxidants on lipid peroxidation in the mammalian eye. Ophthalmic Res 1996;28:184-192.
143. Orhan H, Marol S, Hepsen I F, Sahin G. Effects of some probable antioxidants on selenite-induced cataract formation and oxidative stress-related parameters in rats. Toxicology 1999;139:219-232.
144. Chanvitayapongs S, Draczynska-Lusiak B, Sun A Y. Amelioration of oxidative stress by antioxidants and resveratrol in PC 12 cells. Neuroreport 1997;8:1499-1502.
145. Frankel E N, Waterhouse A L, Kinsella J E. Inhibition of human LDL oxidation by resveratrol. Lancet 1993;341:1103-1104.
146. Shigematsu S, Ishida S, Hara M, et al. Resveratrol, a red wine constituent polyphenol, prevents superoxide-dependent inflammatory responses induced by ischemia/reperfusion, platelet-activating factor, or oxidants. Free Radic Biol Med 2003;34:810-817.
147. Losa G A. Resveratrol modulates apoptosis and oxidation in human blood mononuclear cells. Eur J Clin Invest 2003;33:818-823.
148. Zhuang H, Kim Y S, Koehler R C, Dore S. Potential mechanism by which resveratrol, a red wine constituent, protects neurons. Ann NY Acad Sci 2003;993:276-286.
149. Militante J D, Lombardini J B. Taurine: evidence of physiological function in the retina. Nutr Neurosci 2002;5:75-90.
150. Di Leo M A, Santini S A, Cercone S, et al. Chronic taurine supplementation ameliorates oxidative stress and Na+K+ ATPase impairment in the retina of diabetic rats. Amino Acids 2002;23:401-6.
151. Bantseev V, Bhardwaj R, Rathbun W, et al. Antioxidants and cataract: (cataract induction in space environment and application to terrestrial aging cataract). Biochem Mol Biol Int 1997;42:1189-97.
152. Cubillos S, Fazzino F, Lima L. Medium requirements for neuritic outgrowth from goldfish retinal explants and the trophic effect of taurine. Int J Dev Neurosci 2002;20:607-17.
153. Pasantes-Morales H, Quiroz H, Quesada O. Treatment with taurine, diltiazem, and vitamin E retards the progressive visual field reduction in retinitis pigmentosa: a 3-year follow-up study. Metab Brain Dis 2002;17:183-97.

1. A neuroprotective formulation for lowering non-intraocular pressure-dependent risk factors of glaucoma, the formulation comprising:
ginkgo biloba extract,
resveratrol, and
pycnogenol.

2. A neuroprotective formulation according to claim 1, wherein the ginkgo biloba extract is substantially the standardized extract EGb 761.

3. A neuroprotective formulation according to any one of claims 1-2, further comprising carnitine.

4. A neuroprotective formulation according to any one of claims 1-3, further comprising methylcobalamin.

5. A neuroprotective formulation according to any one of claims 1-4, further comprising coenzyme Q10.

6. A neuroprotective formulation according to any one of claims 1-5, further comprising alpha-lipoic acid.

7. A neuroprotective formulation according to any one of claims 1-6, further comprising salvia miltiorrhiza.

8. A neuroprotective formulation according to any one of claims 1-7, further comprising folic acid.

9. A neuroprotective formulation according to any one of claims 1-8, further comprising ginseng extract.

10. A neuroprotective formulation according to claim 9, wherein the ginseng extract includes at least of one of ginsenoside saponins Rb1 and Rg3.

11. A neuroprotective formulation according to any one of claims 1-10, further comprising fish oil.

12. A neuroprotective formulation according to claim 11, wherein the fish oil includes an omega-3 fatty acid.

13. A neuroprotective formulation according to claim 12, wherein the omega-3 fatty acid includes at least one of docosahexaenoic acid and eicosapentaeneoic acid.

14. A neuroprotective formulation for lowering non-intraocular pressure-dependent risk factors of glaucoma, the formulation comprising:
carnitine,
methylcobalamin, and
coenzyme Q10.

15. A neuroprotective formulation according to claim 14, further comprising ginkgo biloba extract.

16. A neuroprotective formulation according to claim 15, wherein the ginko biloba extract is substantially the standardized extract EBb761.

17. A neuroprotective formulation according to any one of claims 14-16, further comprising resveratrol.

18. A neuroprotective formulation according to any one of claims 14-17, further comprising pycnogenol.

19. A neuroprotective formulation according to any one of claims 14-18, further comprising alpha-lipoic acid.

20. A neuroprotective formulation according to any one of claims 14-19, further comprising salvia miltiorrhiza.

21. A neuroprotective formulation according to any one of claims 14-20, further comprising folic acid.

22. A neuroprotective formulation according to any one of claims 14-21, further comprising ginseng extract.

23. A neuroprotective formulation according to claim 22, wherein the ginseng extract includes at least of one of ginsenoside saponins Rb1 and Rg3.

24. A neuroprotective formulation according to any one of claims 14-23, further comprising fish oil.

25. A neuroprotective formulation according to claim 24, wherein the fish oil includes an omega-3 fatty acid.

26. A neuroprotective formulation according to claim 25, wherein the omega-3 fatty acid includes at least one of docosahexaenoic acid and eicosapentaeneoic acid.

27. A neuroprotective formulation for lowering non-intraocular pressure-dependent risk factors of glaucoma, the formulation comprising:
ginseng extract; and
salvia militiorrhiza.

28. A neuroprotective formulation according to claim 27, wherein the ginseng extract includes at least of one of ginsenoside saponins Rb1 and Rg3.

29. A neuroprotective formulation according to any one of claims 27-28, further comprising alpha-lipoic acid.

30. A neuroprotective formulation according to any one of claims 27-29, further comprising folic acid.

31. A neuroprotective formulation according to any one of claims 27-30, further comprising fish oil.

32. A neuroprotective formulation according to claim 31, wherein the fish oil includes an omega-3 fatty acid.

33. A neuroprotective formulation according to claim 32, wherein the omega-3 fatty acid includes at least one of docosahexaenoic acid and eicosapentaeneoic acid.

34. A neuroprotective formulation according to any one of claims 27-33, further comprising carnitine.

35. A neuroprotective formulation according to any one of claims 27-34, further comprising methylcobalamin.

36. A neuroprotective formulation according to any one of claims 27-35, further comprising coenzyme Q10.

37. A neuroprotective formulation according to any one of claims 27-36, further comprising resveratrol.

38. A neuroprotective formulation according to any one of claims 27-37, further comprising pycnogenol.

39. A neuroprotective formulation according to any one of claims 27-38, further comprising ginkgo biloba extract.

40. A neuroprotective formulation according to claim 39, wherein the ginko biloba extract is substantially the standardized extract EBb761.

41. A neuroprotective formulation for lowering non-intraocular pressure-dependent risk factors of glaucoma, the formulation comprising:
ginko biloba extract; and
salvia militiorrhiza.

42. A neuroprotective formulation according to claim 41, wherein the ginkgo biloba extract is substantially the standardized extract EGb 761.

43. A neuroprotective formulation according to any one of claims 41-42, further comprising alpha-lipoic acid.

44. A neuroprotective formulation according to any one of claims 41-43, further comprising folic acid.

45. A neuroprotective formulation according to any one of claims 41-44, further comprising ginseng extract.

46. A neuroprotective formulation according to claim 45, wherein the ginseng extract includes at least of one of ginsenoside saponins Rb1 and Rg3.

47. A neuroprotective formulation according to any one of claims 41-46, further comprising fish oil.

48. A neuroprotective formulation according to claim 47, wherein the fish oil includes an omega-3 fatty acid.

49. A neuroprotective formulation according to claim 48, wherein the omega-3 fatty acid includes at least one of docosahexaenoic acid and eicosapentaeneoic acid.

50. A neuroprotective formulation according to any one of claims 41-49, further comprising carnitine.

51. A neuroprotective formulation according to any one of claims 41-50, further comprising methylcobalamin.

52. A neuroprotective formulation according to any one of claims 41-51, further comprising coenzyme Q10.

53. A neuroprotective formulation according to any one of claims 41-52, further comprising resveratrol.

54. A neuroprotective formulation according to any one of claims 41-53, further comprising pycnogenol.

55. A neuroprotective formulation for lowering non-intraocular pressure-dependent risk factors of glaucoma, the formulation comprising:
carnitine; and
folic acid.

56. A neuroprotective formulation according to claim 55, further comprising methylcobalamin.

57. A neuroprotective formulation according to any one of claims 55-56, further comprising coenzyme Q10.

58. A neuroprotective formulation according to any one of claims 55-57, further comprising alpha-lipoic acid.

59. A neuroprotective formulation according to any one of claims 55-58, further comprising salvia miltiorrhiza.

60. A neuroprotective formulation according to any one of claims 55-59, further comprising ginseng extract.

61. A neuroprotective formulation according to claim 60, wherein the ginseng extract includes at least of one of ginsenoside saponins Rb1 and Rg3.

62. A neuroprotective formulation according to any one of claims 55-61, further comprising fish oil.

63. A neuroprotective formulation according to claim 62, wherein the fish oil includes an omega-3 fatty acid.

64. A neuroprotective formulation according to claim 63, wherein the omega-3 fatty acid includes at least one of docosahexaenoic acid and eicosapentaeneoic acid.

65. A neuroprotective formulation according to any one of claims 55-64, further comprising ginkgo biloba extract.

66. A neuroprotective formulation according to claim 65, wherein the ginko biloba extract is substantially the standardized extract EBb761.

67. A neuroprotective formulation according to any one of claims 55-66, further comprising resveratrol.

68. A neuroprotective formulation according to any one of claims 55-67, further comprising pycnogenol.

69. A neuroprotective formulation for lowering non-intraocular pressure-dependent risk factors of glaucoma, the formulation comprising:
alpha-lipoic acid;
fish oil; and
pycnogenol.

70. A neuroprotective formulation according to claim 69, wherein the fish oil includes an omega-3 fatty acid.

71. A neuroprotective formulation according to claim 70, wherein the omega-3 fatty acid includes at least one of docosahexaenoic acid and eicosapentaeneoic acid.

72. A neuroprotective formulation according to any one of claims 69-71, further comprising carnitine.

73. A neuroprotective formulation according to any one of claims 69-72, further comprising methylcobalamin.

74. A neuroprotective formulation according to any one of claims 69-73, further comprising coenzyme Q10.

75. A neuroprotective formulation according to any one of claims 69-74, further comprising alpha-lipoic acid.

76. A neuroprotective formulation according to any one of claims 69-75, further comprising salvia miltiorrhiza.

77. A neuroprotective formulation according to any one of claims 69-76, further comprising folic acid.

78. A neuroprotective formulation according to any one of claims 69-77, further comprising ginseng extract.

79. A neuroprotective formulation according to claim 78, wherein the ginseng extract includes at least of one of ginsenoside saponins Rb1 and Rg3.

80. A neuroprotective formulation according to any one of claims 69-79, further comprising ginkgo biloba extract.

81. A neuroprotective formulation according to claim 80, wherein the ginko biloba extract is substantially the standardized extract EBb761.

82. A neuroprotective formulation according to any one of claims 69-81, further comprising resveratrol.

83. A neuroprotective formulation for lowering non-intraocular pressure-dependent risk factors of glaucoma, the formulation comprising:
ginko biloba extract; and
pycnogenol.

84. A neuroprotective formulation according to claim 83, wherein the ginkgo biloba extract is substantially the standardized extract EGb 761.

85. A neuroprotective formulation according to any one of claims 83-84, further comprising carmitine.

86. A neuroprotective formulation according to any one of claims 83-85, further comprising methylcobalamin.

87. A neuroprotective formulation according to any one of claims 83-86, further comprising coenzyme Q10.

88. A neuroprotective formulation according to any one of claims 83-87, further comprising alpha-lipoic acid.

89. A neuroprotective formulation according to any one of claims 83-88, further comprising salvia miltiorrhiza.

90. A neuroprotective formulation according to any one of claims 83-89, further comprising folic acid.

91. A neuroprotective formulation according to any one of claims 83-90, further comprising ginseng extract.

92. A neuroprotective formulation according to claim 91, wherein the ginseng extract includes at least of one of ginsenoside saponins Rb1 and Rg3.

93. A neuroprotective formulation according to any one of claims 83-92, further comprising fish oil.

94. A neuroprotective formulation according to claim 93, wherein the fish oil includes an omega-3 fatty acid.

95. A neuroprotective formulation according to claim 94, wherein the omega-3 fatty acid includes at least one of docosahexaenoic acid and eicosapentaeneoic acid.

96. A neuroprotective formulation for lowering non-intraocular pressure-dependent risk factors of glaucoma, the formulation comprising:
ginko biloba extract; and
resveratrol.

97. A neuroprotective formulation according to claim 96, wherein the ginkgo biloba extract is substantially the standardized extract EGb 761.

98. A neuroprotective formulation according to any one of claims 96-97, further comprising carmitine.

99. A neuroprotective formulation according to any one of claims 96-98, further comprising methylcobalamin.

100. A neuroprotective formulation according to any one of claims 96-99, further comprising coenzyme Q10.

101. A neuroprotective formulation according to any one of claims 96-100, further comprising alpha-lipoic acid.

102. A neuroprotective formulation according to any one of claims 96-101, further comprising salvia miltiorrhiza.

103. A neuroprotective formulation according to any one of claims 96-102, further comprising folic acid.

104. A neuroprotective formulation according to any one of claims 96-103, further comprising ginseng extract.

105. A neuroprotective formulation according to claim 104, wherein the ginseng extract includes at least of one of ginsenoside saponins Rb1 and Rg3.

106. A neuroprotective formulation according to any one of claims 96-105, further comprising fish oil.

107. A neuroprotective formulation according to claim 106, wherein the fish oil includes an omega-3 fatty acid.

108. A neuroprotective formulation according to claim 107, wherein the omega-3 fatty acid includes at least one of docosahexaenoic acid and eicosapentaeneoic acid.

109. A neuroprotective formulation for lowering non-intraocular pressure-dependent risk factors of glaucoma, the formulation comprising:
pycnogenol; and
resveratrol.

110. A neuroprotective formulation according to claim 109, further comprising carnitine.

111. A neuroprotective formulation according to any one of claims 109-110, further comprising methylcobalamin.

112. A neuroprotective formulation according to any one of claims 109-111, further comprising coenzyme Q10.

113. A neuroprotective formulation according to any one of claims 109-112, further comprising alpha-lipoic acid.

114. A neuroprotective formulation according to any one of claims 109-113, further comprising salvia miltiorrhiza.

115. A neuroprotective formulation according to any one of claims 109-114, further comprising folic acid.

116. A neuroprotective formulation according to any one of claims 109-115, further comprising ginseng extract.

117. A neuroprotective formulation according to claim 116, wherein the ginseng extract includes at least of one of ginsenoside saponins Rb1 and Rg3.

118. A neuroprotective formulation according to any one of claims 109-117, further comprising fish oil.

119. A neuroprotective formulation according to claim 118, wherein the fish oil includes an omega-3 fatty acid.

120. A neuroprotective formulation according to claim 119, wherein the omega-3 fatty acid includes at least one of docosahexaenoic acid and eicosapentaeneoic acid.

121. A neuroprotective formulation according to any one of claims 1-120, further comprising green tea catchins.

122. A neuroprotective formulation according to any one of claims 1-121, further comprising quercetin.

123. A neuroprotective formulation according to any one of claims 1-122, further comprising grape seed extract.

124. A neuroprotective formulation according to any one of claims 1-123, further comprising alpha-tocopherol.

125. A neuroprotective formulation according to any one of claims 1-124, further comprising choline.

126. A neuroprotective formulation according to any one of claims 1-125, further comprising glutathione.

127. A neuroprotective formulation according to any one of claims 1-126, further comprising N-acetyl-L cysteine.

128. A neuroprotective formulation according to any one of claims 1-127, further comprising taurine.

129. A neuroprotective formulation according to any one of claims 1-128, further comprising zeaxanthine.

130. A neuroprotective formulation according to any one of claims 1-129, further comprising lutein.

131. A neuroprotective formulation according to any one of claims 1-130, further comprising citicoline.

132. A neuroprotective formulation according to any one of claims 1-131, further comprising at least one of vitamin A, copper, magnesium, selenium, and zinc.

133. A neuroprotective formulation according to any one of claims 1-132, wherein the formulation includes at least one pill.

134. A neuroprotective formulation according to claim 133, wherein the at least one pill is a plurality of pills with at least one pill having a different composition than one other pill.

135. A neuroprotective formulation according to claim 133, wherein the at least one pill is a plurality of pills with at least one pill having a component in a differing quantity than one other pill.

136. A method of preventing the progression of glaucoma in a patient, the method comprising the steps of:
providing a neuroprotective formulation according to any one of claims 1-132; and
administering the neuroprotective formulation to the patient to reduce the risk of one or more non-intraocular pressure-dependent factors of glaucoma in a patient.

137. A method according to claim 136, wherein the neuroprotective formulation is contained in a dosage formulation.

138. A method according to claim 136, wherein administering the neuroprotective formulation includes administering the formulation on a scheduled basis.

139. A method according to claim 138, wherein administering the neuroprotective formulation includes administering one or more particular formulations according to the scheduled basis.

140. A method of preventing the progression of glaucoma in a patient, the method comprising the steps of:
providing a neuroprotective formulation according to any one of claims 1-132; and
administering the neuroprotective formulation to the patient to strengthen the resistance of damage to nerve cells.
